(19) United States
(12) Patent Application Publication
(10) Pub. No.: US 2005/0215763 A1
(43) Pub. Date: Sep. 29, 2005

(54) Method for producing gelatin

(76) Inventor: Per Sjorup, Randers (DK)

Correspondence Address: STITES & HARBISON PLLC, 1199 NORTH FAIRFAX STREET, SUITE 900, ALEXANDRIA, VA, 22314, US

(21) Appl. No.: 10/512,097

(22) PCT Filed: Apr. 15, 2003
(86) PCT No.: PCT/DK03/00255

§ 371 Date: May 17, 2005

(30) Foreign Application Priority Data
Apr. 22, 2002 (DK) PA 2002 00599

(51) Int. Cl. C07K 14/78

(52) U.S. Cl. 530/354

(57) ABSTRACT

Gelatin is produced by defatting and chopping rind, hydrolysing with acid, neutralising, and extracting with water. By defatting the rind before hydrolysing, the yield of high Bloom gelatin is typically 50% higher than previously. Furthermore, the gelatin thus produced has a higher Bloom strength.

### TECHNICAL FIELD

This invention relates to a method for producing gelatin, and gelatin produced by the method.

### BACKGROUND ART

Conventionally gelatin is prepared from rind, usually from swine, by first chopping the rind with the accompanying fat layer into pieces of e.g. 60 by 100 mm, hydrolysing the chopped rind with acid, e.g. hydrochloric acid, in e.g. 20 to 24 hours, neutralising and extracting with water, first at 50° C. and thereafter at successively rising temperature, the best gelatin quality, high Bloom, being obtained at 50° C. Usually, a yield of 20% to 40% of the gelatin present in the rind is obtained in high Bloom quality with a Bloom strength of 280 g.

### DISCLOSURE OF THE INVENTION

It has now been found that a better product and a higher yield is obtained when the rind is defatted before it is hydrolysed. Thus the yield of high Bloom gelatin is typically 50% to 60% of the gelatin present in the rind, i.e. about 50% higher than by using the conventional method, and the produced gelatin has a higher strength than the gelatin produced by the conventional method.

Accordingly, the method of the invention is characterised by that the rind is defatted before the hydrolysis, and the gelatin of the invention is characterised by being produced by the method of the invention.

Preferably, the defatting is carried out in a continuous process.

If the rind is sufficiently comminuted, e.g. in pieces of 1 mm, the hydrolysis may also be carried out continuously.

The defatting can be carried out by the addition of steam and hot water to melt off the fat from the rind. Thus, U.S. Pat. No. 2,748,152 discloses the preparation of defatted rind by heating chopped rind together with water under melting off fat, separation of the products and purification of the defatted rind by washing with water in a centrifuge. However, the rind defatted in this way is not used for the production of gelatine.

U.S. Pat. No. 5,877,287 discloses defatting in relation to gelatin production in column 4, lines 17 to 21, in example 1, and in claim 11. In the examples, bones are used as the starting material. The object of the defatting disclosed in the patent is to avoid a particular process step which formerly was necessary for the complete demineralisation of the bone material before the hydrolysis, cf. column 6, lines 23 to 28.

The method of the invention differs from the method of said U.S. patent by using another starting material, namely rind instead of bones, and by another object of the defatting, namely obtaining gelatin of a higher quality and higher yield, whereas the object of the method of the U.S. patent is to facilitate the hydrolysis. By the method of the U.S. patent the defatting does not seem to lead to a higher quality gelatin.

The invention is further illustrated by the working example below which merely should act as an illustration and by no way as a limitation of the invention.

### EXAMPLE

6,660 kg of rind, chopped into pieces of appr. 5 mm, are defatted with steam and hot water in a continuous process to a fat content of 2% and are carried to a 10 m³ reactor. This gives 5,000 kg of defatted rind. 5,000 liters of water are filled into the reactor, and 37% hydrochloric acid is added to obtain a pH of 2. After 17 hours the hydrochloric acid is removed and the hydrolysed rind is neutralised with water to obtain a pH of 4. Thereafter the hydrolysed and neutralised rind is extracted with 15 m³ of water at 50° C. for 50 minutes.

After evaporation and drying 432 kg of gelatin are obtained with high Bloom quality, which amount to 43% of the gelatin present in the rind. The gelatin has a Bloom strength of 335 g.

If the defatting is omitted, the yield is 315 kg corresponding to only 30% of the gelatin present in the rind and the Bloom strength of that gelatin is only 280 g.

1. A method for producing gelatin wherein rind is chopped (cut), hydrolyzed by acid, neutralized, and extracted with water, characterized by that the rind is defatted before the hydrolysis.

2. The method of claim 1, characterized by that the defatting is carried out in a continuous process.

3. The method of claim 1, characterized by that the rind is defatted to a fat content of 2% to 3%.

4. The method claim 1, characterized by that the rind is comminuted into pieces of 5 mm or less before the hydrolysis.

5. Gelatin, characterized by being produced by the method of claim 1.
